(19) United States
(12) Patent Application Publication
(10) Pub. No.: US 2014/0236247 A1
(43) Pub. Date: Aug. 21, 2014

(54) SURGICAL IMPLANT SYSTEM AND METHOD

(71) Applicant: WARSAW ORTHOPEDIC, INC., Warsaw, IN (US)

(72) Inventor: William Alan Rezach, Atoka, TN (US)

(73) Assignee: WARSAW ORTHOPEDIC, INC., Warsaw, IN (US)

(21) Appl. No.: 13/772,072

(22) Filed: Feb. 20, 2013

(51) Int. Cl. A61B 17/88; A61B 17/86

(52) U.S. Cl. CPC A61B 17/8875; A61B 17/8615
USPC 606/308; 606/104

(57) ABSTRACT

A surgical implant driver includes a member defining an engagement portion including a wall having a circumference and a plurality of lobes disposed thereabout. The lobes are curved toward an interior of the circumference and are connected by a plurality of arcuate portions that are curved toward the interior of the circumference.

### TECHNICAL FIELD

The present disclosure generally relates to medical devices for the treatment of musculoskeletal disorders, and more particularly to a surgical system for delivering and/or fastening implants with a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, lam inectomy and implantable prosthetics. As part of these surgical treatments, implants such as bone fasteners, connectors, plates and vertebral rods are often used to provide stability to a treated region. These implants can redirect stresses away from a damaged or defective region while healing takes place to restore proper alignment and generally support the vertebral members. For example, rods and plates may be attached via the fasteners to the exterior of two or more vertebral members. This disclosure describes an improvement over these prior art technologies.

### SUMMARY

In one embodiment, in accordance with the principles of the present disclosure, a surgical implant system is provided. The surgical implant system includes a member defining an engagement portion including a wall having a circumference and a plurality of lobes disposed thereabout. The lobes are curved toward an interior of the circumference and are connected by a plurality of arcuate portions that are curved toward the interior of the circumference.

### BRIEF DESCRIPTION OF THE DRAWINGS

The present disclosure will become more readily apparent from the specific description accompanied by the following drawings, in which:

FIG. 1 is a side view, in part cross section, of one particular embodiment of components of a surgical implant system in accordance with the principles of the present disclosure, with the components separated;

FIG. 2 is a side view of a component of the surgical implant system shown in FIG. 1;

FIG. 3 is an end view of the component shown in FIG. 2;

FIG. 4 is an enlarged end view of Detail A shown in FIG. 3;

FIG. 5 is a break away perspective view of the component shown in FIG. 2;

FIG. 6 is a side view of a component of the surgical implant system shown in FIG. 1;

FIG. 7 is a cross section view of the component shown in FIG. 6 taken along lines B-B;

FIG. 8 is an end view of the component shown in FIG. 6;

FIG. 9 is a side view of the system shown in FIG. 1, with the components engaged;

FIG. 10 is a cross section view of the system shown in FIG. 1 taken along lines C-C in FIG. 9;

FIG. 11 is a cross section view of one particular embodiment of a surgical implant system in accordance with the principles of the present disclosure; and

FIG. 12 is an end view of the component shown in FIG. 6.

Like reference numerals indicate similar parts throughout the figures.

### DETAILED DESCRIPTION

The exemplary embodiments of a surgical implant system are discussed in terms of medical devices for the treatment of musculoskeletal disorders and more particularly, in terms of a surgical implant system for delivering and/or fastening implants with a surgical site and a method for treating a spine. In one embodiment, the system includes a driver that includes a series of spaced apart convexly curved first surfaces or lobes and a series of spaced apart curved second surfaces or arcuate portions connecting and alternating with the first surfaces. In one embodiment, the bit includes a series of spaced apart convexly curved first surfaces or lobes and a series of spaced apart convexly curved second surfaces or arcuate portions connecting and alternating with the first surfaces. In some embodiments, the bit may be utilized with any driver having a solid cross section to provide advantages over standard hexalobe bits including, for example, higher torque values, backwards compatibility with other types of drivers, such as, for example T25 drivers and ease of manufacture.

In one embodiment, the bit is configured for disposal in a socket of an implant, such as, for example, a bone screw or set screw having a drive interface to engage the driver with the implant. The lobes of the bit are configured to engage the lobes of the drive interface and the arcuate portions of the bit are configured to engage the arcuate portions of the drive interface when the lobes of the bit engage the lobes of the drive interface. The drive interface includes a series of spaced apart concavely curved first surfaces or lobes and a series of spaced apart concavely curved second surfaces or arcuate portions connecting and alternating with the first surfaces. In some embodiments, the drive interface may be formed using seven straight drill features.

In some embodiments, the surgical implant system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the surgical implant system of the present disclosure may be employed with other osteal and bone related applications, including those associated with diagnostics and therapeutics. In some embodiments, the disclosed surgical implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The surgical implant system of the present disclosure may also be alternatively employed with procedures for treating the lumbar, cervical, thoracic, sacral and pelvic regions of a spinal column. The surgical implant system of the present disclosure may also be used on animals, bone models and other non-living substrates, such as, for example, in training, testing and demonstration.

The surgical implant system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

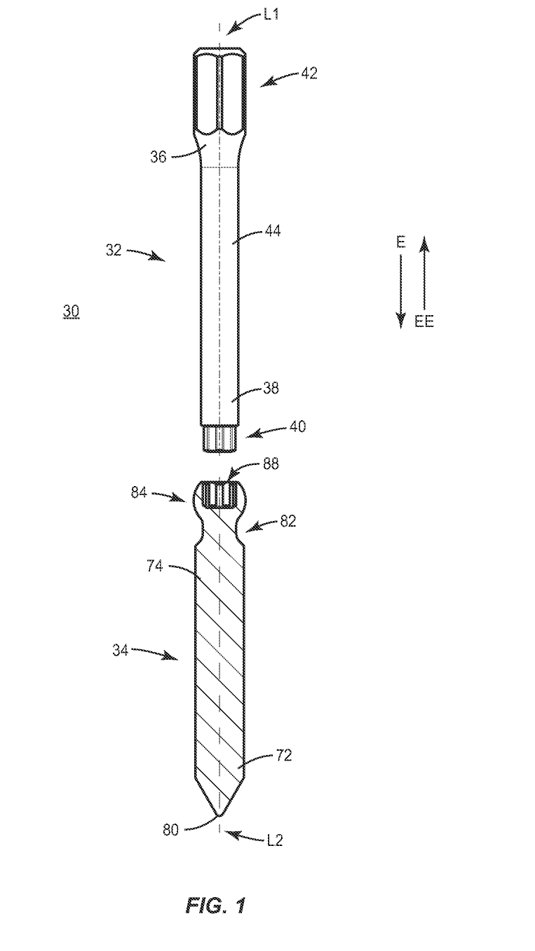

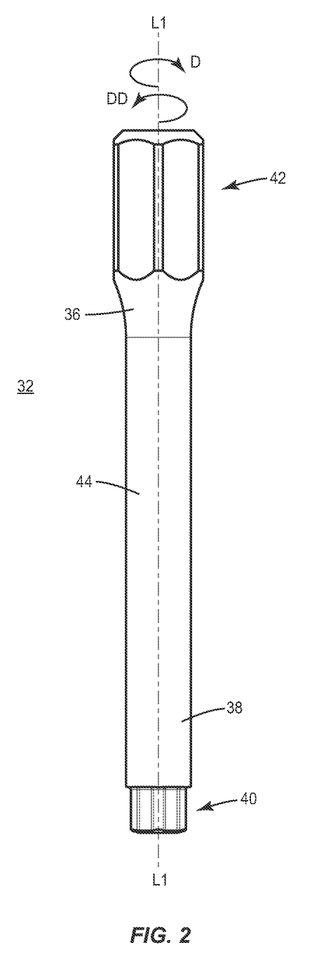

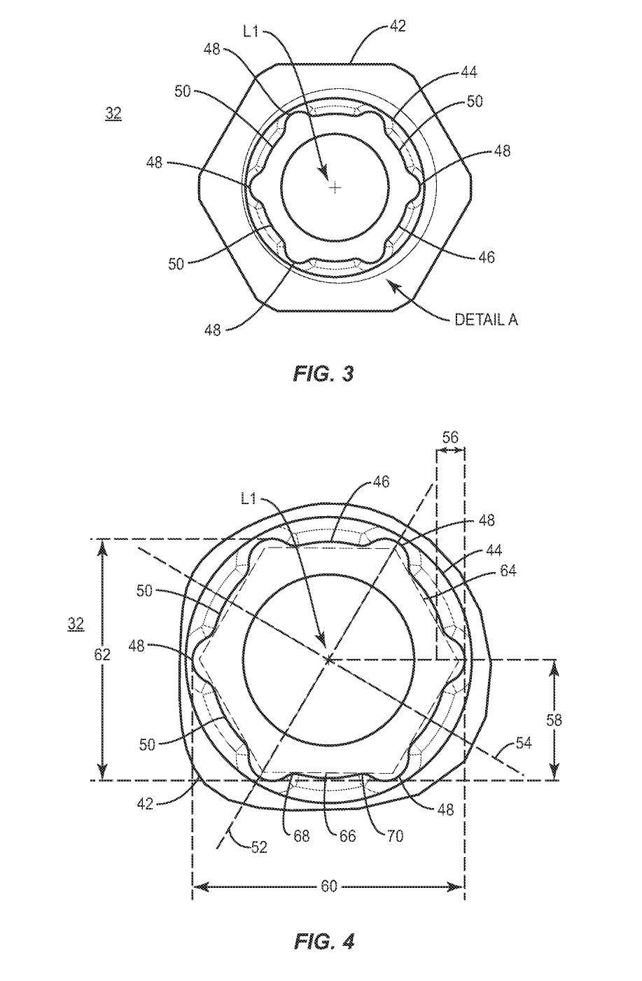

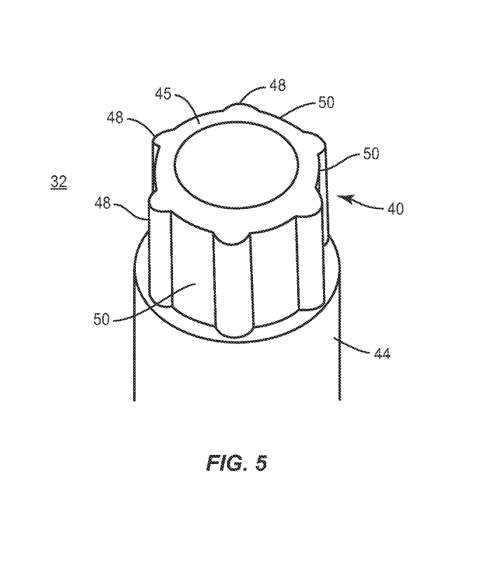

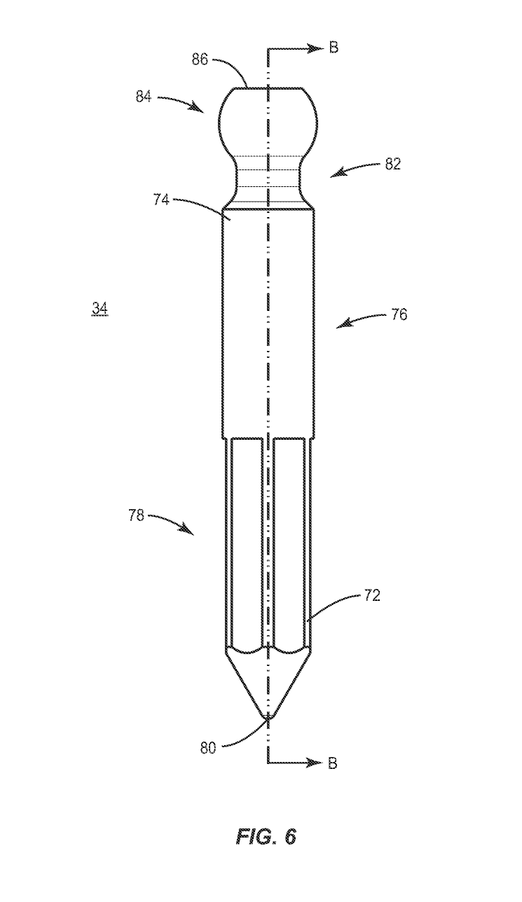

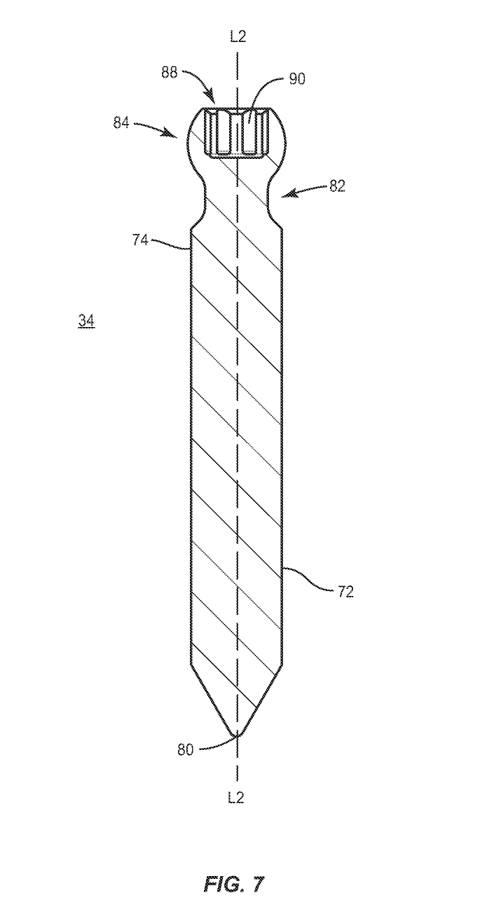

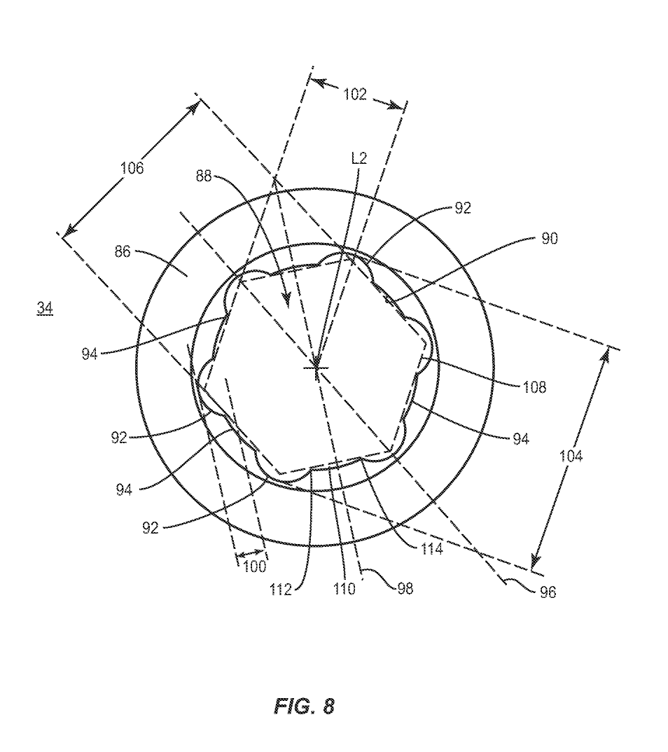

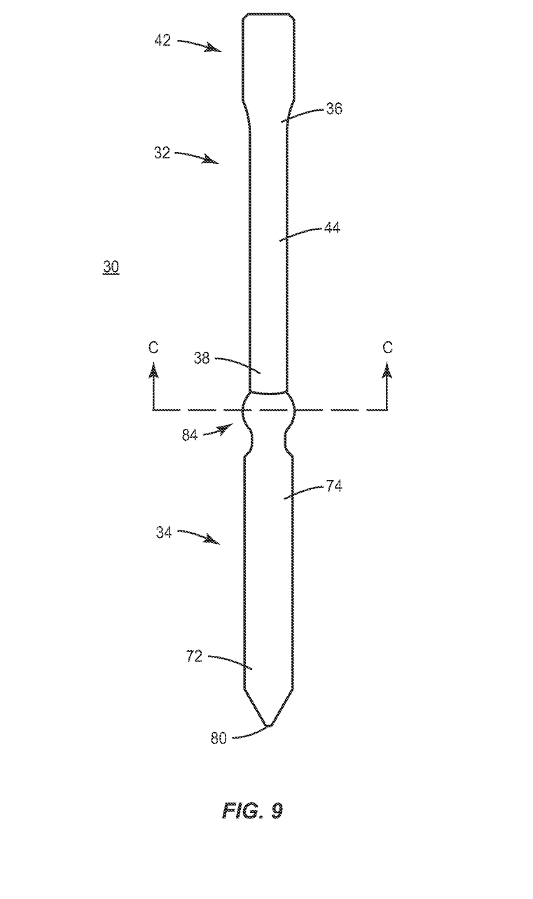

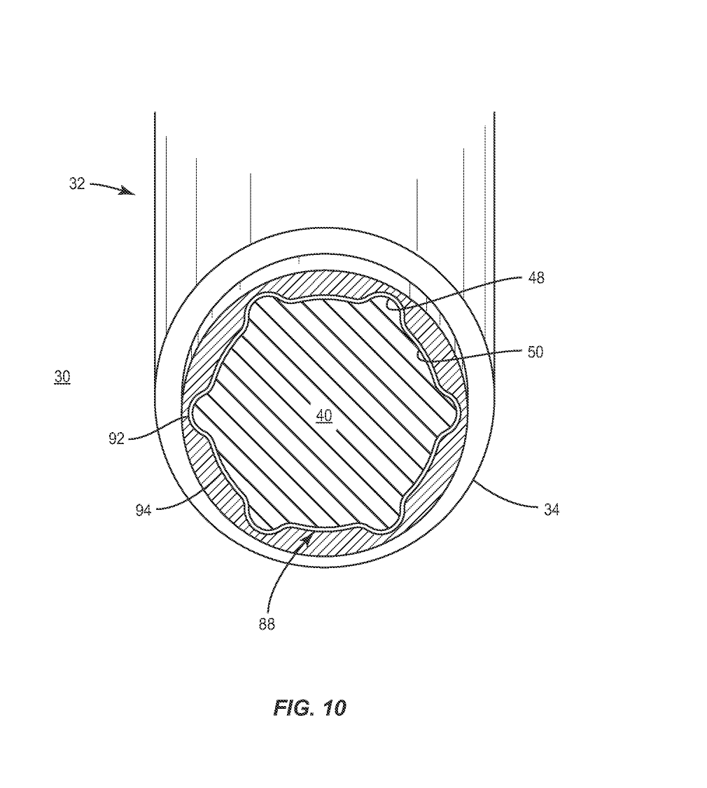

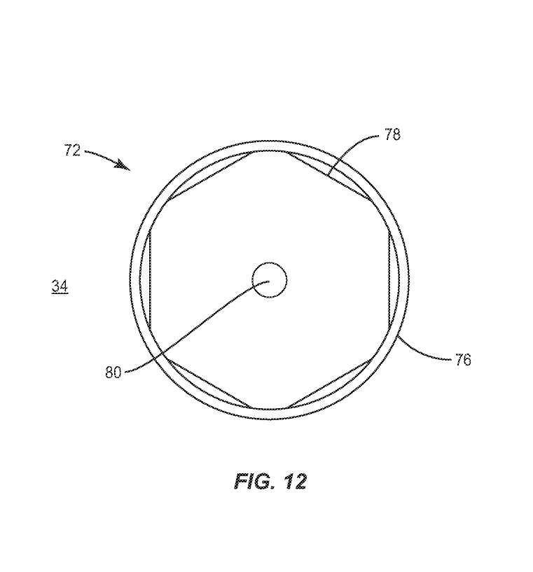

The following discussion includes a description of a surgical implant system including a driver and a bone fastener, related components and methods of employing the surgical implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-12, there is illustrated components of a surgical implant system 30 including a member, such as, for example, a surgical implant driver 32 and a fastener, such as, for example, a bone fastener 34, in accordance with the principles of the present disclosure.

The components of surgical implant system 30 can be fabricated from biologically acceptable materials suitable for medical applications, including metals, synthetic polymers, ceramics and bone material and/or their composites, depending on the particular application and/or preference of a medical practitioner. For example, the components of surgical implant system 30, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of surgical implant system 30 may have material composites, including the above materials, to achieve various desired characteristics such as strength, rigidity, elasticity, compliance, biomechanical performance, durability and radiolucency or imaging preference. The components of surgical implant system 30, individually or collectively, may also be fabricated from a heterogeneous material such as a combination of two or more of the above-described materials. The components of surgical implant system 30 may be monolithically formed, integrally connected or include fastening elements and/or instruments, as described herein.

Surgical implant system 30 is employed, for example, with an open or mini-open, minimal access and/or minimally invasive including percutaneous surgical technique to deliver and fasten an implant, such as, for example, a bone fastener at a surgical site within a body of a patient, for example, a section of a spine. In one embodiment, the components of surgical implant system 30 are configured to fix a spinal rod, connector and/or plate to a spine via a bone fastener for a surgical treatment to treat various spine pathologies, such as those described herein.

Driver 32 extends along a first longitudinal axis L1 between a first end 36 and a second end 38 defining an engagement portion 40. End 36 includes a drive portion 42 configured to engage an actuator, such as, for example, a surgical instrument, powered drill, hand drill, driver or other tool to rotate driver 32 in the direction shown by arrow D and/or the direction shown by arrow DD. In one embodiment, portion 42 has a hexagonal cross sectional configuration and is configured to engage a correspondingly shaped portion of the actuator. In some embodiments, portion 42 may include an oval, oblong, triangular, square or polygonal cross sectional configuration configured engage a correspondingly shaped portion of the actuator. In one embodiment, end 36 includes an interchangeable driving handle removably connected to portion 42 such that torque applied manually or by motorized means to the handle is transmitted to portion 42.

End 36 is tapered between portion 42 and a shaft 44 such that portion 42 has a width that is greater than a width of shaft 44. Shaft 44 has a cylindrical cross sectional configuration and a uniform width along the length of shaft 44. In some embodiments, end 36 may be variously configured and dimensioned between portion 42 and a shaft 44, such as, for example, irregular, uniform, non-uniform, staggered, tapered, consistent or variable, depending on the requirements of a particular application. In some embodiments, shaft 44 may be variously configured and dimensioned, such as, for example, planar, concave, convex or polygonal, depending on the requirements of a particular application.

Portion 40 includes a beveled edge 45 and a wall 46 having a circumference and a plurality of lobes 48 disposed thereabout. Lobes 48 are uniformly spaced apart about the circumference of wall 46. Lobes 48 curve toward an interior of the circumference of wall 46. Adjacent lobes 48 are connected by an arcuate portion 50. Lobes 48 merge smoothly and tangentially with portions 50. Portions 50 curve toward the interior of the circumference of wall 46. Lobes 48 are convexly curved between adjacent portions 50 and portions 50 are convexly curved between adjacent lobes 48. Portion 40 has a solid cross section between opposite lobes 48 and opposite portions 50. The arcuate portions 50 all have a common center, and the portions 50 all lie along the same circumference. The centers of the arcs defining the lobes 48 are within the circumference of the arcuate portions 50.

In one embodiment, portion 40 includes six lobes 48 that are uniformly spaced apart about the circumference of wall 46 such that lobes 48 define corners of a modified hexalobe bit and portions 50 defines sides thereof. It is envisioned that portion 40 may include two or more lobes 48 connected by an equal number of portions 50. In one embodiment, lobes 48 are semicylindrical or substantially semicylindrical. In some embodiments, lobes 48 and/or portions 50 may be variously configured and dimensioned, such as, for example, polygonal, irregular, uniform, non-uniform, staggered, tapered, consistent or variable, depending on the requirements of a particular application.

Lobes 48 each have a center or axis of curvature 52. Portions 50 each have a center or axis of curvature 54. Axes 52 are disposed on the same side of wall 46 with respect to axis L1. Axes 52 are transverse to axes 54 and axis L1. Lobes 48 each have a radius of curvature 56 that is less than a radius of curvature 58 of each of portions 50. In one embodiment, radius 56 is between about ⅓ and about ⅙ of radius 58. In one embodiment, radius 56 is between about ⅓ and about ¼ of radius 58.

Portion 40 has a maximum diameter 60 defined by the distance between opposite lobes 48 and a minimum diameter 62 defined by the distance between opposite portions 50. Shaft 44 has a diameter that is greater than diameter 60. Lobes 48 extend from axis L1 a first distance and portions 50 extend from axis L1 a second distance, the second distance being less than the first distance. The difference between the first and second distances is equal to a third distance defined by one half of the difference between diameter 60 and diameter 62. The third distance defines the difference between the maximum radial dimension of lobes 48 and the maximum radial dimension of portions 50, both dimensions being measured from axis L1. In one embodiment, the third distance is equal to radius 56. In one embodiment, the third distance is greater than radius 56.

Axes 52 are located at the apices of a regular hexagon, indicated by dotted line 64. There is a gap 66 between portions 50 and line 64 such that each portion 50 engages line 64 at a first interface 68 between the portion 50 and a first lobe 48 and at a second interface 70 between the portion 50 and a lobe 48 adjacent to the first lobe.

Fastener 34 extends along a second longitudinal axis L2 between a distal portion 72 configured to penetrate tissue and a proximal portion 74. Portion 72 includes a first section 76 having a cylindrical cross section and a uniform diameter and a second section 78 having a hexagonal cross section and a uniform diameter that is less than the diameter of section 76. Section 76 is tapered between section 78 and a tip 80. In some embodiments, section 76 and/or section 78 may be variously configured and dimensioned, such as, for example, planar, concave, convex, polygonal, irregular, uniform, non-uniform, staggered, tapered, consistent or variable, depending on the requirements of a particular application. In one embodiment, portion 72 has a uniform diameter along sections 76, 78.

In one embodiment, fastener 34 includes an outer surface including a thread form configured to penetrate tissue, such as, for example, bone, to fix fastener 34 in such tissue. It is contemplated that the thread form on the outer surface of fastener 34 may extend along sections 76, 78. It is further contemplated that the thread form on the outer surface of fastener 34 may extend along section 76 without extending along any portion of section 78. It is envisioned that all or only a portion of the outer surface of fastener 34 may have various surface configurations, such as, for example, rough, threaded, arcuate, undulating, porous, semi-porous, dimpled, polished and/or textured according to the requirements of a particular application.

Portion 74 includes a neck 82 extending from section 76 and a substantially spherical head 84 extending from neck 82. Head 84 has a maximum diameter that is substantially equivalent to the diameter of section 76 and neck 82 has a diameter that is less than the diameter of section 76 and the maximum diameter of head 84 such that fastener 34 is tapered between head 84 and neck 82 and between section 76 and neck 82.

Head 84 includes a planar proximal face 86 extending perpendicular to axis L2 and having a socket 88 disposed therein extending parallel to axis L2 and defining a drive interface configured for engagement with portion 40. In some embodiments, face 86 and/or socket 88 may be disposed at alternate orientations relative to axis L2, such as, for example, transverse, perpendicular and/or other angular orientations such as acute or obtuse, according to the requirements of a particular application. In some embodiments, face 86 may be variously configured and dimensioned, such as, for example, concave, convex, irregular, uniform, non-uniform, staggered, tapered, consistent or variable, depending on the requirements of a particular application.

Socket 88 includes an inner surface 90 having a circumference and a plurality of lobes 92 disposed thereabout. Lobes 92 are uniformly spaced apart about the circumference of surface 90. Lobes 92 curve toward an interior of the circumference of surface 90. Adjacent lobes 92 are connected by an arcuate portion 94. Lobes 92 merge smoothly and tangentially with portions 94. Portions 94 curve toward the interior of the circumference of surface 90. Lobes 92 are concavely curved between adjacent portions 94 and portions 94 are concavely curved between adjacent lobes 92. Socket 88 has a hollow cross section between opposite lobes 92 and opposite portions 94 configured for disposal of portion 40 to engage driver 32 with fastener 34 such that rotation of driver 32 in the direction shown by arrow D or the direction shown by arrow DD also causes rotation of fastener 34 in the direction shown by arrow D or the direction shown by arrow DD.

In one embodiment, socket 88 includes six lobes 92 that are uniformly spaced apart about the circumference of surface 90 such that lobes 92 define corners of a modified hexalobe socket and portions 94 defines sides thereof. It is envisioned that socket 88 may include two or more lobes 92 connected by an equal number of portions 94. In one embodiment, lobes 92 are semicylindrical or substantially semicylindrical. In some embodiments, lobes 92 and/or portions 94 may be variously configured and dimensioned, such as, for example, polygonal, irregular, uniform, non-uniform, staggered, tapered, consistent or variable, depending on the requirements of a particular application.

Lobes 92 each have a center or axis of curvature 96 and portions 94 each have a center or axis of curvature 98 such that axes 96, 98 are disposed on the same side of surface with respect to axis L2. Axes 96 are located at the apices of a regular hexagon, indicated by dotted line 108. Axes 96 are transverse to axes 98 and axis L2. Lobes 92 each have a radius of curvature 100 that is less than a radius of curvature 102 of each of portions 94. In some embodiments, radius 100 is between about ⅓ and about ⅙ of radius 102 or between about ⅓ and about ¼ of radius 102. Radius 100 is greater than radius 56 and radius 102 is greater than radius 58. In one embodiment, radius 100 is equal to radius 56 and radius 102 is equal to radius 58. There is a gap 110 between portions 94 and line 108 such that each portion 94 engages line 108 at a first interface 112 between the portion 94 and a first lobe 92 and at a second interface 114 between the portion 94 and a lobe 92 adjacent to the first lobe 92.

In some embodiments, socket 88 may have a depth defined by the distance between face 86 and a bottom portion of socket 88 that is equal to radius 56 or radius 102. In one embodiment, the depth of socket 88 is less than radius 56 and/or radius 102. In one embodiment, the bottom portion of socket 88 is chamfered such that edge 45 of driver 32 engages the bottom portion of socket 88 when portion 40 is disposed within socket 88.

Socket 88 has a maximum diameter 104 defined by the distance between opposite lobes 92 and a minimum diameter 106 defined by the distance between opposite portions 94. Face 86 has a diameter that is greater than diameter 104. Diameter 104 is greater than diameter 60 and diameter 106 is greater than diameter 62 such that portion 40 may be inserted into socket 88 to engage driver 32 with fastener 34. When portion 40 is inserted into socket 88, axes L1, L2 are coaxial. In one embodiment, diameter 104 is substantially equivalent to diameter 60 and diameter 106 is substantially equivalent to diameter 62 such that lobes 48 closely engage lobes 92 and portions 50 closely engage portions 94 when portion 40 is inserted into socket 88.

Lobes 92 extend from axis L2 a fourth distance and portions 94 extend from axis L2 a fifth distance, the fifth distance being less than the fourth distance. The difference between the fourth and fifth distances is equal to a sixth distance defined by one half of the difference between diameter 104 and diameter 102. The sixth distance defines the difference between the maximum radial dimension of lobes 92 and the maximum radial dimension of portions 94, both dimensions being measured from axis L2. In one embodiment, the sixth distance is equal to radius 100. In one embodiment, the sixth distance is greater than radius 100.

In one embodiment, as shown in FIG. 10, the configuration of portion 40 corresponds to the configuration of socket 88 such that portion 40 closely engages socket 88 and socket 88 closely engages portion 40. Radius 56 is substantially equivalent to radius 100 and radius 58 is substantially equivalent to radius 102. Diameter 60 is less than diameter 104 and diameter 62 is less than diameter 106. The difference between diameters 60, 104 is proportional to the difference between diameters 62, 106 such that portion 40 closely matches socket 88 when portion 40 is disposed within socket 88 to engage driver 32 with fastener 34. That is, there is no gap between portion 40 and socket 88 or there is very small gap between portion 40 and socket 88 that has a uniform width.

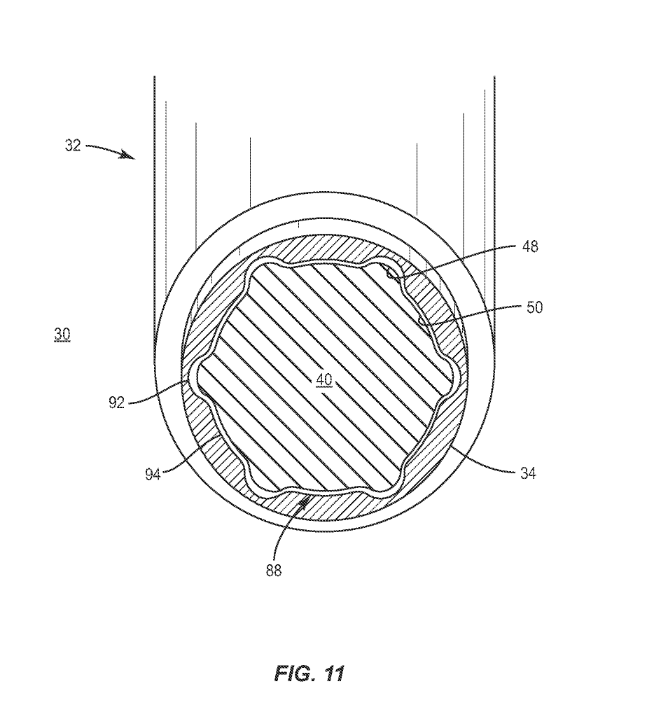

In one embodiment, as shown in FIG. 11, lobes 48 have a radius of curvature that is substantially less than a radius of curvature of lobes 92, while portions 50 have a radius of curvature that is substantially equivalent to a radius of curvature of portions 94 such that there is no gap between portions 50, 94, or only a small gap between portions 50, 94, and a larger gap between lobes 48, 92.

In assembly, operation and use, an implant system, similar to system 30 described herein, is employed with a surgical procedure for treatment of a spinal disorder affecting a section of a spine of a patient, as discussed herein. For example, the spinal implant system can be used with a surgical procedure for treatment of a condition or injury of an affected section of the spine including vertebrae (not shown). In some embodiments, one or all of the components of system 30 can be delivered or implanted as a pre-assembled device or can be assembled in situ. System 30 may be completely or partially revised, removed or replaced.

For example, system 30 can be employed with a surgical treatment of an applicable condition or injury of an affected section of a spinal column and adjacent areas within a body, such as, for example, vertebrae. It is envisioned that system 30 may be employed with one or a plurality of vertebra. To treat a selected section of the vertebrae, a medical practitioner obtains access to a surgical site including the vertebrae in any appropriate manner, such as through incision and retraction of tissues. In some embodiments, system 30 can be used in any existing surgical method or technique including open surgery, mini-open surgery, minimally invasive surgery and percutaneous surgical implantation, whereby the vertebrae are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of system 30. A preparation instrument (not shown) can be employed to prepare tissue surfaces of the vertebrae, as well as for aspiration and irrigation of a surgical region according to the requirements of a particular surgical application.

A pilot hole or the like is made in a selected vertebra of the vertebrae for receiving a fastener 34. System 30 is disposed adjacent the vertebrae at a surgical site and the components of system 30 including driver 32, are manipulable to drive, torque, insert or otherwise connect fastener 34 to the vertebra, according to the particular requirements of the surgical treatment. For example, lobes 48 are aligned with lobes 92 and portions 50 are aligned with portions 94 with portion 40 spaced apart from socket 88. Driver 32 is translated axially relative to fastener 34 in the direction shown by arrow E such that lobes 48 engage lobes 92 and portions 50 engage portions 94, as shown in FIG. 10, to matingly and releasably fix driver 32 with fastener 34. Fastener 34 may then be inserted into the vertebra with driver 32, for example, by rotating driver 32 in the direction shown by arrow D or the direction shown by arrow DD, which causes rotation of fastener 34 in the direction shown by arrow D or the direction shown by arrow DD. As fastener 34 rotates in the direction shown by arrow D and the direction shown by arrow DD, fastener 34 translates within the vertebra in the direction shown by arrow E or arrow EE. Upon completion of a surgical procedure, driver 32 may be disengaged from fastener 34, and the non-implanted components, including driver 32 may be removed from the surgical site and the incision closed.

In some embodiments, one or a plurality of bone fasteners may be employed with a single vertebral level or a plurality of vertebral levels. In some embodiments, the bone fasteners may be engaged with vertebrae in various orientations, such as, for example, series, parallel, offset, staggered and/or alternate vertebral levels. System 30 can be used with various bone fasteners, mono-axial screws, sagittal angulation screws, fixed screws, uni-planar screws, pedicle screws or multi-axial screws used in spinal surgery. In some embodiments, fastener 34 may include a set screw configured to be threaded into an opening in a screw, such as, for example, a bone screw to maintain an implant, such as, for example, a vertebral rod, within a cavity of the bone screw by engaging the rod such that the rod applies a force to the cavity to fix the rod relative to the bone screw.

In one embodiment, system 30 includes an agent, which may be disposed, packed or layered within, on or about the components and/or surfaces of system 30. In some embodiments, the agent may include bone growth promoting material, such as, for example, bone graft to enhance fixation of the bone fasteners with the vertebrae. In some embodiments, the agent may include one or a plurality of therapeutic agents and/or pharmacological agents for release, including sustained release, to treat, for example, pain, inflammation and degeneration. The components of system 30 can be made of radiolucent materials such as polymers. Radiomarkers may be included for identification under x-ray, fluoroscopy, CT or other imaging techniques. In some embodiments, the use of microsurgical and image guided technologies may be employed to access, view and repair spinal deterioration or damage, with the aid of system 30.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical implant system comprising:
a member defining a first longitudinal axis and an engagement portion including a wall having a circumference and a plurality of lobes disposed thereabout, the lobes being curved toward an interior of the circumference and each lobe having a center, the lobes being connected by a plurality of arcuate portions, the arcuate portions being curved toward the interior of the circumference, wherein the centers of each lobe are within the circumference of the wall.

2. A surgical implant system as recited in claim 1, further comprising:
a socket defining a second longitudinal axis and a drive interface including an inner surface having a circumference and a plurality of lobes disposed thereabout, the lobes of the drive interface being curved toward an interior of the circumference of the inner surface, the lobes of the engagement portion being configured to engage the lobes of the drive interface, the lobes of the drive interface being connected by a plurality of arcuate portions, the arcuate portions of the drive interface being curved toward the interior of the circumference of the inner surface, the arcuate portions of the engagement portion being configured engage the arcuate portions of the drive interface when the lobes of the engagement portion engage the lobes of the drive interface.

3. A surgical implant system as recited in claim 1, wherein the plurality of lobes comprises six lobes.

4. A surgical implant system as recited in claim 2, wherein the plurality of lobes of the engagement portion include six lobes and the plurality of lobes of the drive interface include six lobes.

5. A surgical implant system as recited in claim 1, wherein the lobes are convexly curved between adjacent arcuate portions and the arcuate portions are convexly curved between adjacent lobes.

6. A surgical implant system as recited in claim 2, wherein the lobes of the engagement portion are convexly curved between adjacent arcuate portions of the engagement portion, the arcuate portions of the engagement portion are convexly curved between adjacent lobes of the engagement portion, the lobes of the drive interface are concavely curved between adjacent arcuate portions of the drive interface and the arcuate portions of the drive interface are concavely curved between adjacent lobes of the drive interface.

7. A surgical implant system as recited in claim 2, wherein the lobes of the engagement portion and the lobes of the drive interface are semicylindrical.

8. A surgical implant system as recited in claim 1, wherein the lobes each define a first axis of curvature and the arcuate portions each define a second axis of curvature, the first and second axes of curvature being transverse to one another and the first longitudinal axis.

9. A surgical implant system as recited in claim 1, wherein the lobes each define a first axis of curvature and the arcuate portions each define a second axis of curvature, the first and second axes of curvature being disposed on the same side of the wall with respect to the first longitudinal axis.

10. A surgical implant system as recited in claim 9, wherein the lobes of the drive interface each define a third axis of curvature and the arcuate portions of the drive interface each define a fourth axis of curvature, the third axes of curvature are disposed on a side of each lobe opposite the second longitudinal axis, the fourth axes of curvature being disposed on a side of each arcuate portion opposite the second longitudinal axis.

11. A surgical implant system as recited in claim 1, wherein the lobes each have a radius of curvature that is less than a radius of curvature of each of the arcuate portions.

12. A surgical implant system as recited in claim 1, wherein the engagement portion includes a major diameter defined by a distance between opposite lobes and a minor diameter defined by a distance between opposite arcuate portions, the minor diameter being less than the major diameter.

13. A surgical implant system as recited in claim 12, wherein the drive interface includes a major diameter defined by a distance between opposite lobes of the drive interface and a minor diameter defined by a distance between opposite arcuate portions of the drive interface, the minor diameter of the drive interface being less than the major diameter of the drive interface, the major diameter of the drive interface being greater than the major diameter of the engagement portion and the minor diameter of the drive interface being greater than the minor diameter of the engagement portion.

14. A surgical implant system as recited in claim 1, wherein the engagement portion has a solid cross section between opposite lobes and opposite arcuate portions.

15. A surgical implant system comprising:
a member defining a first longitudinal axis and an engagement portion including a wall having a circumference and a plurality of lobes disposed thereabout, the lobes being connected by a plurality of arcuate portions, the lobes being convexly curved between adjacent arcuate portions, the arcuate portions being convexly curved between adjacent lobes, the lobes each having a radius of curvature that is less than a radius of curvature of each of the arcuate portions; and
a socket defining a second longitudinal axis and a drive interface including an inner surface having a circumference and a plurality of lobes disposed thereabout such, the lobes of the engagement portion being configured to engage the lobes of the drive interface, the lobes of the drive interface being connected by a plurality of arcuate portions, the arcuate portions of the engagement portion being configured engage the arcuate portions of the drive interface when the lobes of the engagement portion engage the lobes of the drive interface, the lobes of the drive interface being concavely curved between adjacent arcuate portions of the drive interface, the arcuate portions of the drive interface being concavely curved between adjacent lobes of the drive interface.

16. A surgical implant system as recited in claim 15, wherein the plurality of lobes of the engagement portion include six lobes and the plurality of lobes of the drive interface include six lobes.

17. A surgical implant system as recited in claim 15, wherein the lobes of the engagement portion each define a first axis of curvature and the arcuate portions of the engagement portion each define a second axis of curvature, the first and second axes of curvature being disposed on the same side of the wall with respect to the first longitudinal axis.

18. A surgical implant system as recited in claim 17, wherein the lobes of the drive interface each define a third axis of curvature and the arcuate portions of the drive interface each define a fourth axis of curvature, the third axes of curvature are disposed on a side of each lobe opposite the second longitudinal axis, the fourth axes of curvature being disposed on a side of each arcuate portion opposite the second longitudinal axis.

19. A surgical implant system as recited in claim 15, wherein the lobes of the engagement portion each have a radius of curvature that is less than a radius of curvature of each of the arcuate portions of the engagement portion and the lobes of the drive interface each have a radius of curvature that is less than a radius of curvature of each of the arcuate portions of the drive interface.

20. A surgical implant system comprising:
a driver defining a first longitudinal axis and an engagement portion including a wall having a circumference and six convexly curved lobes disposed thereabout each having a semicylindrical configuration, the lobes being connected by six convexly curved arcuate portions, the lobes each have a radius of curvature that is less than a radius of curvature of each of the arcuate portions, the engagement portion having a solid cross section between opposite lobes and opposite arcuate portions; and
a bone fastener extending along a second longitudinal axis between a distal portion configured to penetrate tissue and a proximal portion including a socket defining a drive interface, the drive interface including an inner surface having a circumference and six concavely curved lobes disposed thereabout each having a semicylindrical configuration, the lobes of the engagement portion being configured to engage the lobes of the drive interface, the lobes of the drive interface being connected by six concavely curved arcuate portions, the lobes of the drive interface each have a radius of curvature that is less than a radius of curvature of each of the arcuate portions of the drive interface such that the arcuate portions of the engagement portion engage the arcuate portions of the drive interface when the lobes of the engagement portion engage the lobes of the drive interface,
wherein the radius of curvature of the lobes of the engagement portion is greater than the radius of curvature of the lobes of the drive interface and the radius of curvature of the arcuate portions of the engagement portion is greater than the radius of curvature of the lobes of the drive interface.
